(19) United States
(12) Patent Application Publication
(10) Pub. No.: US 2022/0409787 A1
(43) Pub. Date: Dec. 29, 2022

(54) VALVE ISLAND FOR A HYDRAULIC ASSEMBLY FOR A DIALYSIS MACHINE

(71) Applicant: B. Braun Avitum AG, Melsungen (DE)

(72) Inventors: Rolf Bertram, Bergheim (DE); Peter Maenz, Melsungen (DE)

(21) Appl. No.: 17/780,114

(22) PCT Filed: Nov. 25, 2020
(86) PCT No.: PCT/EP2020/083282

§ 371 Date: May 26, 2022

(30) Foreign Application Priority Data
Nov. 27, 2019 (DE) 10 2019 132 178.6

(51) Int. Cl. A61M 1/14

(52) U.S. Cl. CPC A61M 1/1565

(57) ABSTRACT

A valve island for a hydraulic assembly and a hydraulic assembly featuring a valve island. The valve island and the hydraulic assembly are used with a dialysis machine. The valve island has a main part in which at least one fluid flow duct is formed. The main part is produced by additive manufacturing or injection molding. The hydraulic assembly includes a support on which at least one valve island is arranged directly or indirectly.

### CROSS-REFERENCE TO RELATED APPLICATIONS

This application is the United States national stage entry of International Application No. PCT/EP2020/083282, filed Nov. 25, 2020, and claims priority to German Application No. 10 2019 132 178.6. The contents of International Application No. PCT/EP2020/083282 and German Application No. 10 2019 132 178.6 are incorporated by reference herein in their entireties.

### FIELD

The present disclosure relates to a valve island for a, or respectively, of a hydraulic assembly in a dialysis machine, a hydraulic assembly with a valve island, and a dialysis machine with a corresponding hydraulic assembly comprising the valve island.

### BACKGROUND

Dialysis devices/dialysis machines in medical technology usually include a hydraulic system or a hydraulic assembly in which valve units are used in which (electromagnetically switched) valves can selectively block or release the inputs and outputs of fluid line components of the dialysis machine in order to form a desired fluid flow path.

Conventional valve units for dialysis machines, such as those described in patent specification EP 895787, use a basic structure in which a balancing device of the dialysis machine is connected to the hydraulic system via solenoid valves. The solenoid valves serve as shut-off valves (on/off valves) for the inlets and outlets of the balance chambers of the balancing device. A central control unit drives the solenoid valves to form a desired fluid flow path.

However, the disadvantage of this solution is that the valve unit has many individual components and the assembly can reach a correspondingly large installation size.

In other solutions in medical technology, valve units of this type use central mounting sheets on which solenoid valves are fixed to form a central portion or valve island in the hydraulic system. According to prior art, for example, an insertion sheet is used on which the solenoid valve(s) is (are) mounted via a screw connection.

The disadvantage of these solutions, however, is that tools are always required for assembly and repair in order to fasten or disconnect the valves. This leads to an increase in the required installation space as well as an increase in the assembly steps.

### SUMMARY

The objective of the disclosure is therefore to provide a valve island for a generic hydraulic assembly/hydraulic system and a hydraulic assembly/hydraulic system with a valve island, in particular of or for a dialysis machine, with which the assembly effort can be reduced while at the same time reducing the installation size of the valve island.

Accordingly, the core of the present disclosure basically consists in the arrangement of a valve island with a base body, in/on which at least one fluid line (tube/fluid flow passage) is formed integrally/in one piece of material, at the at least one, preferably both axial ends (front side) of which in each case a hydraulic/pneumatic component, in particular a valve and further preferably a solenoid valve, is arranged/mounted via which a fluid connection between a respective (preferably end-side/front-side) fluid line opening and a component-side/valve-side branch line/passage can be opened and/or closed. The integral fluid line or fluid line formed in one piece of material with the base body furthermore has a number (at least one or more) of branch connections (spouts) which are spaced apart in the axial direction of the fluid line and, if applicable, are arranged circumferentially offset and are formed in one piece of material with the base body as well. Preferably, the spouts are fluid-separated from each other (e.g. by a partition wall in the integral fluid line) in such a way that in each case one spout is assigned only to one (solenoid) valve arranged at the front side, so that the two (solenoid) valves arranged axially at the end side are quasi media-separated. Furthermore, a further port may be formed in one piece of material on the integral fluid line, for example for the optional connection of an additional (solenoid) valve. Finally, a number (at least one) of mounting/holding means (projections, hooks, tabs, etc.) may be formed in one piece of material on the base body, which are provided and configured for fastening the base body to a separate holder/platform (not belonging to the valve island).

This constructive design of the valve island according to the disclosure makes it possible to configure its base body (including all components provided in one piece according to the above design) in a single section/one piece of material in a single manufacturing step, in particular using an injection molding or 3D printing process (rapid prototyping process). The connections on the integral fluid line that are (simultaneously) formed in the process allow (subsequent) assembly of (solenoid) valves, external fluid lines, temperature/electrical conductivity/pressure/fluid flow sensors and similar hydraulic/pneumatic components without the use of tools. Consequently, the manufacture of the valve island designed in this way is inexpensive and simple. Since the mounting means are preferably also integrated on the base body, installation of the valve island in a machine, preferably a dialysis machine, is quick.

According to one aspect of the invention, the valve island for a or of a hydraulic assembly is provided for a or of an extracorporeal blood treatment machine, preferably a dialysis machine. The valve island comprises the base body in which at least the one, in particular rigid, fluid flow passage (integral fluid line) is formed. The base body is, as configured above, preferably manufactured by additive manufacturing, or injection molding.

By using 3D printing or injection molding processes, the base body can be manufactured in a single step. The one or more integral fluid flow passages can be designed variably. In injection molding, interchangeable inserts can be used for this purpose. The at least one integral fluid flow passage is preferably provided rigidly and can therefore also assume a load-bearing function. Due to this self-supporting function, a conventional housing can be dispensed with for the valve island or the hydraulic assembly.

In a further development of the valve island, at least one valve mounting portion is formed/arranged (in one piece of material with the base body), which, as configured above, is provided at at least one axial end/end portion of the integral fluid flow passage. The valve mounting portion may be formed, for example, as a coil element, in particular a coil carrier for a solenoid valve, a solenoid valve itself, a valve seat, a control edge, a simple screw thread or bayonet fitting, or any other type of a valve mounting portion.

The valve mounting section or the solenoid valve may also be detachably/rotatably arranged on the base body, wherein the valve mounting portion is preferably manufactured additively or further preferably via injection molding. Different variants of the solenoid valve, the valve seat, etc., can be manufactured using an injection molding tool. In particular, a valve mounting portion formed in one piece of material on the base body (for example, in the form of the coil mounting body described above) reduces the number of individual parts that have to be attached to the base body. In other words, the number of individual parts or components required is reduced. In addition, this saves the assembly time required for this purpose. Furthermore, the installation space provided for this purpose, which has to be planned for an entire hydraulic assembly, can be efficiently reduced.

The valve mounting portion may be arranged together with a valve seat and perform the function of a media-separated valve to ensure contamination safety of the extracorporeal fluid circuit.

In a further development of the valve island, the at least one solenoid valve arranged on the front side of the integral fluid flow passage is held on the base body so that it can be rotated axially in steps of less than or equal to 45° in particular. This allows the cable routing for the valve portion to be individually adapted or oriented and also optimized in terms of material requirements and accessibility in the event of assembly and service. The valve is preferably mechanically secured to prevent it from turning automatically. In addition, cable length can be saved.

In a further development of the valve island, at least one, in particular hollow-cylindrical, coupling receptacle (referred to above as port) is formed in one piece with the base body, via which a (further) hydraulic component, in particular radially sealing/radially tight, is fluidically and form-fittingly connectable, is in particular connected, to the at least one fluid flow passage. The coupling receptacle may be injection molded onto the base body via injection molding or 3D printed. Radial-tight adaptation of other hydraulic components to the base body, such as a (further) valve, a tube coupling, a conductivity measurement sensor, a pressure measurement sensor, a temperature sensor, a pressure regulator, a throttle, a pressure reduction valve or the like, is thus possible.

In a further development of the valve island, the coupling receptacle (port), which is in particular in the form of a hollow cylinder, has on the circumferential side, in particular on the inner circumferential side, at least one, preferably a plurality, of depressions (longitudinal grooves) into which an element of a hydraulic component adapted for this purpose is insertable, is in particular inserted. In other words, the coupling receptacle has a connection portion formed with a certain profile, into/onto which the hydraulic component (further valve) can be inserted/fitted in a rotationally fixed manner as well as possibly in a predetermined rotational position with respect to the connection portion. In this way, rotation of the hydraulic elements relative to the coupling receptacle can be avoided. Such an anti-rotation device, for example via serrations (grooves), enables improved orienting of the hydraulic component with regard to the routing of the hydraulic lines, in particular hoses, and the routing of the cables or the wiring harness of the dialysis machine. The anti-rotation device can alternatively have different geometries with different dimensions. This can be used to implement an error prevention principle, such as the ‘poka-yoke principle’. In one case, for example, only one (single) orientation of the components may be possible. For axial securing of the hydraulic element in the coupling receptacle, a clip-shaped cotter pin can preferably be provided, which can be inserted into the coupling receptacle transversely to the latter and thus prevents axial removal/extraction of the hydraulic element out of/from the coupling receptacle.

In a further development of the valve island, at least one spout is formed in one piece of material with the base body, via which an external fluidic line is fluidically connectable, is in particular connected, to the at least one integral fluid flow passage. The at least one spout may be molded onto the base body via injection molding or printed on via 3D printing.

The diameter of the at least one spout can be variable. The at least one spout is provided for connecting or plugging on fluidic lines with an inner diameter of in particular 2 to 6 mm. The fluidic line is in particular a tube, but may alternatively be a pipe. The base body preferably has 2 to 3 or 4 to 6 spouts. The number of spouts can be extended to a maximum of 7.

In a further development of the valve island, at least one fluidic and possibly also mechanical connecting portion is formed integrally with the base body, via which a preferably sensory component, in particular a hydraulic sensor component, is fluidically and possibly also mechanically connectable, is in particular connected. This enables radially sealed/radially tight adaptation or connection of sensor components to the base body, such as a conductivity measurement sensor, a pressure measurement sensor, a temperature sensor or the like. Alternatively, other hydraulic components such as a (further) valve, a tube coupling, a pressure regulator, a throttle, a pressure reducing valve or the like can be connected.

In a further development of the valve island, at least one connecting portion is formed on the base body, via which the valve island is connectable, is in particular connected, to a connecting portion of the hydraulic assembly or of a mounting adapter matched thereto.

In a further development of the valve island, at least two recesses, in particular two bores, are formed on the base body through which fixing means, in particular cable ties, are passable. Alternatively, a different number of bores, for example four bores, can be formed/provided on the base body to enable fixing adapted to the corresponding circumstances. The fixing means are intended to provide strain relief for at least one cable or cable harness.

In a further development of the valve island, at least one receptacle, in particular a plug window, is formed in the base body, in which a plug housing is receivable, is in particular received. Alternatively, two plug windows may be formed on the base body, each of which receives a plug housing. The plug window enables a plug housing of a load line or a plug housing of a sensor line to be received.

In a further development of the valve island, the hydraulic component is a valve, in particular a solenoid valve, which is connected fluidically and form-fittingly (on the front side) to the at least one integrated fluid flow passage, in particular radially tightly, via the coupling receptacle arranged/formed on the end side of the integrated fluid flow passage. In a further development of the valve island, the valve has a valve seat which is manufactured in particular by additive manufacturing or injection molding.

In a further development of the valve island, the valve has a coupling portion, in particular a frustoconical coupling portion, which is inserted into the coupling receptacle described above and is axially fastened therein via a mounting element, in particular a mounting clip (split pin as defined above). The coupling portion may be manufactured by injection molding or additive manufacturing. By inserting the frustoconical coupling portion into the coupling receptacle, a fluidic, radially tight connection is established. Via the fluidic, radially tight connection, the fluid flow channels of the base body and the valve are connected. The mounting element (split pin) can be manufactured via injection molding or additive manufacturing, or can be produced as a stamped bent part or as a wire bent part.

In a further development of the valve island, the aforementioned coupling portion has an element, in particular a projection, adapted for the at least one depression of the coupling receptacle. The projection of the coupling portion engages in the depression of the coupling receptacle. This prevents the valve portion from rotating in relation to the base body.

In a further development of the valve island, the coupling portion has a sealing element, in particular an O-ring, at a section, in particular at the circumference of a tip end, for sealing a formed fluid flow passage.

In a further development of the valve island, the valve has a valve portion, in particular a coil element. The valve portion can be designed axially or angled in relation to the coupling portion, for example angled by about 90°.

In a further development of the valve island, the valve portion is axially rotatable in steps, in particular of less than or equal to 45°.

In a further development of the valve island, the valve has at least one spout (valve-side branch passage as defined above) via which an external fluid line, in particular a pipe or tube, is fluidically connectable, is in particular connected, to the at least one integral fluid flow passage.

The at least one spout may be designed axially or angled in relation to the coupling portion and/or the valve portion, for example angled by about 90°.

The valve may have two or more spouts that are formed axially or angled relative to each other, for example angled by about 90°.

In an alternative further development of the valve island, the valve has a fluidic and possibly also mechanical connecting portion via which a component, in particular a hydraulic component, is fluidically and possibly also mechanically connectable, in particular connected, to the valve.

Examples of a hydraulic component are, for example, another valve, in particular a snap-in valve, a tube coupling, a conductivity measuring sensor, a pressure measuring sensor, a temperature sensor, a pressure regulator, a throttle, a pressure reducing valve or similar sensory component.

The fluidic and possibly also mechanical connecting portion may be designed axially or angled, for example angled by about 90°, in relation to the coupling portion and/or the valve portion and/or the at least one spout.

In a further development of the valve island, a mixing element for mixing fluids is integrated in a fluid flow passage of the/a valve. The valve is preferably a hydraulic check valve or a hydraulic solenoid valve.

In an alternative further development of the valve island, a mixing element is integrated in a valve seat of the valve. The valve is preferably a hydraulic solenoid valve, in particular the snap-in valve.

In one further embodiment of the valve island, the mixing element is tubular and extends along its length in the axial direction within the fluid flow passage of the valve.

In a further embodiment of the valve island, the hydraulic component is a mixing element that is fluidically and form-fittingly connected to a fluid flow passage or the at least one fluid flow passage via the/a coupling receptacle.

In a further development of the valve island in which the hydraulic component is a mixing element, the mixing element preferably has a full-cylinder shape. A plurality of flow passages are formed within the solid cylindrical mixing element in the direction of extension of the mixing element. The flow passages are arranged parallel to each other and parallel to a central axis of the mixing element to form a multiple passage for a fluid flowing therethrough. The flow passages extend in the axial direction of the fluid flow passage of the valve and/or fluid flow direction.

In a particularly preferred further embodiment of the valve island, a portion of the plurality of flow passages, in particular eight, are arranged in close proximity to the outer circumference of the mixing element in the circumferential direction at uniform intervals. Another part of the plurality of flow passages, in particular four, are evenly spaced in the immediate vicinity of the central axis in the circumferential direction. The radial distances of the one part of the plurality of flow passages from the central axis are uniform in each case. Likewise, the radial distances of the other part of the plurality of flow passages from the central axis are uniform.

In a further embodiment of the valve island, the cross-sectional shape of one part of the plurality of flow passages is different from the cross-sectional shape of the other part of the plurality of flow passages. The cross-sectional shape of the one portion of the plurality of flow passages is preferably circular. The cross-sectional shape of the other part of the plurality of flow passages is preferably polygonal, in particular hexagonal.

In a further development of the valve island, the mixing element, in particular a frustoconical coupling portion, is inserted into the coupling receptacle and is secured therein via a mounting element, in particular a mounting clip.

In a further development of the valve island, the mixing element is manufactured by additive manufacturing or injection molding.

In a further development of the valve island, the mixing element is manufactured in one piece with the coupling portion by injection molding or additive manufacturing. The mixing element may serve as a first component, for example as a pre-molded part, in order to subsequently be connected, in particular overmolded, in one piece with a second component, in particular the coupling portion, by injection molding. The material of the first component and the second component may be different.

By inserting the frustoconical coupling portion into the coupling receptacle, a fluidic, radially tight connection is established. Via the fluidic, radially tight connection, the fluid flow channels of the base body and the mixing element are connected.

The mixing element may be directly integrated into a sub-assembly which comprises, for example, a valve, in particular a check valve, a mounting clip and a conductivity measuring sensor.

The mixing element may be secured against rotation about the longitudinal/central axis of the fluid flow passage of the valve by an anti-rotation device.

Another aspect of the invention is a hydraulic assembly for one or more extracorporeal blood treatment machines comprising a carrier having indirectly or directly arranged thereon at least one valve island according to a first aspect.

The carrier is suitable for modular assembly of at least one component, but preferably a plurality of components. The carrier may be a tower, in particular an assembly tower.

In a further development of the hydraulic assembly, the carrier has, in particular only, one surface or plane on which the one or more valve islands and/or other components is or are arranged. This has the advantage that during maintenance or repair of the hydraulic assembly, there are no components blocking the way, which may have to be dismantled in order to access a particular valve island and/or other component.

In a further development of the hydraulic assembly, the at least one valve island and/or other components in the common surface or plane or assembly plane are mountable or dismountable from a common side.

In a further development of the hydraulic assembly, the carrier has at least one adapted connecting portion that is connected to the connecting portion of the base body, in particular without tools.

This connection can be realized in particular according to the keyhole principle, in which an at least male connection element can be inserted (hooked) into an at least female connection receptacle. However, other tool-free connection variants such as a plug-in principle or the like are also possible.

In a further development of the hydraulic assembly, it has a mounting adapter with an adapted connecting portion that is connected to the connecting portion of the base body, in particular without tools, and has at least one secondary connecting portion that is connected to the carrier.

This connection can also be realized in particular according to the keyhole principle, in which an at least male connection element can be inserted (hooked) into an at least female connection receptacle. However, other tool-free connection variants are also possible, such as a plug-in principle, hook-in principle or the like, as already configured above. The secondary connecting portion of the mounting adapter can be a force-fit or tool-free connection to the carrier.

In a further development of the hydraulic assembly, the mounting adapter is a spacer, via which the base body is arranged approximately parallel to the surface or respectively a mounting angle, via which the base body is arranged at an angle to the surface or respectively the plane.

In a further development of the hydraulic assembly, the connection of the valve island to the carrier or the mounting adapter is locked, in particular via a locking element. The locking element can be an expansion clip, wedge pin or the like.

In a further development of the hydraulic assembly, the carrier is an assembly tower. The assembly tower has a mounting plate or the like that serves as a mounting surface or mounting level for the at least one valve island and/or other hydraulic components. The mounting plate may be provided in a U-shaped frame or a box-shaped housing. The mounting plate may be arranged centrally and parallel between the front and rear walls of the housing, so that a front and rear space is formed which is bounded by the mounting plate. Further valve islands or other hydraulic components may be mounted or suspended on the inner surfaces formed in the respective space, preferably without the use of tools.

In a further development of the hydraulic assembly, the mounting angle is mounted on an insertion sheet that is suitable for insertion or sliding into the hydraulic assembly, in particular the assembly tower.

In a further development of the hydraulic assembly, the latter has at least one sub-assembly which is arranged indirectly or directly on the carrier, in particular by hanging and/or screwing, in deviation from the valve island(s).

The sub-assembly forms a further mounting surface or plane which is planar to the mounting surface or plane of the carrier. On the mounting surface or plane of the carrier, for example, a flat recess can be provided at the edge into which the sub-assembly is or will be inserted in a planar manner.

In a further development of the hydraulic assembly, the sub-assembly has at least one balance chamber. Hydraulic components such as valves, in particular solenoid valves, can be connected to the balance chamber. The connection can be fastened via a mounting element, in particular a mounting clip. The balance chambers can be manufactured additively or via injection molding.

### BRIEF DESCRIPTION OF THE DRAWING FIGURES

The objects, aspects and advantages of the present invention stated above are further illustrated by the following detailed description of the accompanying drawing.

FIG. 1 is a schematic representation of one side of a valve island according to a first configuration example of the present disclosure;

FIG. 2 is a schematic representation of the other side of the valve island according to the first configuration example of the present disclosure;

FIG. 3 is a schematic view of several embodiments a) to e) of a snap-in valve connectable to the valve island, comprising a coupling portion arranged axially with respect to a coil element;

FIG. 4 is a schematic view of several variations a) to e) of the snap-in valve connectable to the valve island, which has a coupling portion angled at 90° to the coil element;

FIG. 5 is a schematic representation of the individual parts of a configuration of the valve island according to the first configuration example with the snap-in valve, a mounting clip, an expansion clip and a connecting portion of the carrier;

FIG. 6 is a magnified representation of a coupling receptacle of the valve island with a mounting clip according to the first configuration example;

FIG. 7 is a schematic representation of a front side of a valve island according to a second configuration example of the present disclosure;

FIG. 8 is a schematic representation of a mounting side of a valve island according to the second configuration example of the present disclosure;

FIG. 9 is a schematic representation of the individual parts of a configuration of the valve island according to the second configuration example with snap-in valves, an expansion clip, a mounting adapter and a connecting portion of the carrier;

FIG. 10 is a schematic representation of a front view of an overall configuration of the second configuration example of the valve island;

FIG. 11 is a schematic representation of a hydraulic assembly with balance chambers and the valve island according to the second configuration example; and

FIG. 12 is a schematic representation of the valve island according to the first configuration example mounted on a mounting angle attached to an insertion sheet.

FIG. 13 is a schematic representation of the individual parts of a configuration of a sub-assembly with a mixing element.

FIG. 14 is a schematic representation of the individual parts of a further configuration of a sub-assembly with a modification of the mixing element.

FIG. 15 is a schematic representation of a further modification of a mixing element.

FIG. 16 is a cross-sectional view of the further modification of the mixing element, which is connected in one piece to a coupling portion.

### DETAILED DESCRIPTION

Configuration examples of the present disclosure are described below based on the accompanying figures.

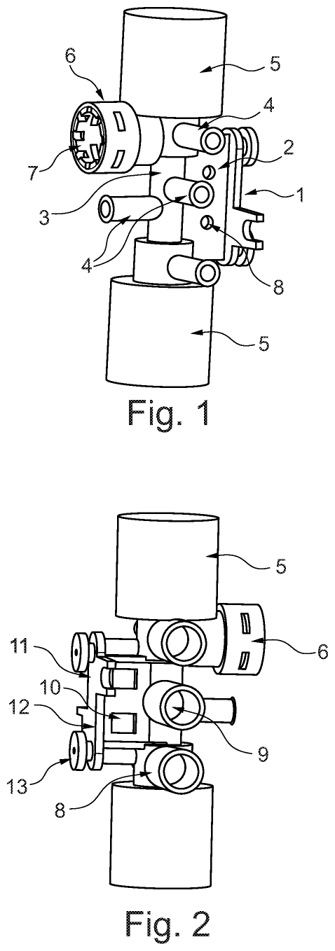

FIG. 1 shows a schematic representation of a longitudinal side of a valve island 1 (perspective view) according to a first configuration example of the present disclosure.

The valve island 1 has a base body 2. The base body 2 is manufactured using additive manufacturing or injection molding. The base body 2 comprises (has) a fluid flow passage 3 which is integral/formed in one piece with the base body 2 (internal, rigid) and thus fulfills a self-supporting function of the valve island 1. The base body 2 furthermore has a plurality of spouts or connection nozzles 4 integral/formed in one piece with the base body 2, which are connected to the fluid flow passage 3, preferably in a T-shaped (fluidically connected) manner, and are provided and configured for the connection of external hydraulic lines or tubes. The spouts 4 are preferably molded onto the base body 2 and point radially outward from the fluid flow passage 3.

In/on the fluid flow passage 3, a respective axial valve seat (not shown) is preferably provided or formed within the two axial ends/end portions of the fluid flow passage 3 or at the two axial ends/end portions of the fluid flow passage 3. Each valve seat is opened and/or closed by a movable valve element (valve piston), which is driven by a coil element (electric valve coil) 5 arranged at the base body 2 or fluid flow passage 3, respectively. The coil elements 5 of the two valves arranged axially at the ends are located axially outside at the axial ends/end portions of the fluid flow passage 3. The coil elements 5 are held on the base body 2 rotatably in 45° degree steps or alternatively in steps smaller than 45° about their axis, i.e. about the longitudinal axis of the fluid flow passage 3.

The base body 2 also has a hollow cylindrical coupling receptacle (port) 6 at/in the area of the valve seat, which is suitable for fluidic and form-fitting (mechanical) connection with other hydraulic components. The coupling receptacle 6 is preferably molded onto the base body 2 and points radially (preferably in a T-shape) outward from the fluid flow passage 3. The hollow-cylindrical coupling receptacle 6 has depressions 7 on the inner circumference at preferably equal circumferential distances, which are suitable and provided for receiving an element of another hydraulic component adapted thereto. The base body 2 also has (through) bores 8 which extend completely through the base body 2 laterally next to the fluid flow passage 3 and through which fixing means such as screws, rivets or bands (not shown) are insertable.

In other words, the valve island according to the disclosure consists of the base body 2, which comprises both the fluid flow passage 3 and a (block-like) portion with the bores 8 formed therein in one piece of material. The fluid flow passage 3 has two axially spaced ends/end portions to which the aforementioned solenoid valves are preferably mounted so as to be rotatable about the longitudinal axis of the passage. Each valve has an electrically activatable valve coil, via which a respective movable valve body is movable for opening and closing the valve, so as to fluidly connect and/or fluidly disconnect the fluid flow passage 3 with a branch line. The valve seat, which is openable and closable by the valve body, is formed either directly on the front side of the fluid flow passage 3 or integrally in the solenoid valve. As an alternative to the rotatability of the valve-internal valve/solenoid coil described above, this (or its receiving housing) can also be formed integrally with the base body 2.

FIG. 2 is a schematic representation of the other side of the valve island 1 with respect to FIG. 1, according to the first configuration example of the present disclosure. On this other side of the base body 2, three fluidic connecting portions 9 are formed which are suitable for the connection/coupling of, for example, sensors or display instruments. The connecting portions 9 are shown in the form of connection nozzles, which are preferably oriented in a T-shape with respect to the fluid flow passage 3. The base body 2 further comprises plug windows 10, which are suitable and provided for receiving/passing wiring or the electrical load line for the valve coils and which are placed adjacent to the fluid flow passage 3.

The other side of the valve island 1 shown in FIG. 2 shows a connecting portion or mounting flange 11 in the area of the aforementioned block-like portion of the base body 2, wherein the connecting portion/mounting flange 11 is formed in one piece of material on the base body 2. The connecting portion 11 is suitable and provided for (mechanically) connecting the valve island 1 to a connecting portion (mounting flange) of a hydraulic assembly or a mounting adapter matched thereto. The connecting portion 11 preferably consists of a connecting base plate 12, which is oriented in the longitudinal direction of the valve island 1 parallel to the fluid flow passage 3 of the base body 2. At the longitudinal ends of the connecting base plate 12 spaced along the fluid flow passage 3, two male connection elements 13 (positioning bases/hanging pins) are formed, which are suitable and provided for insertion or hanging in respective female connection receptacles (tabs/bores), for example of the hydraulic assembly. A web of the male connection element 13 protrudes from the connecting base plate 12 to terminate in a head element having a multiple of the diameter of the web. The head element spans a plane parallel to the plane of the connecting base plate 10—i.e. it is plate-shaped—and thus forms an undercut corresponding to the hook.

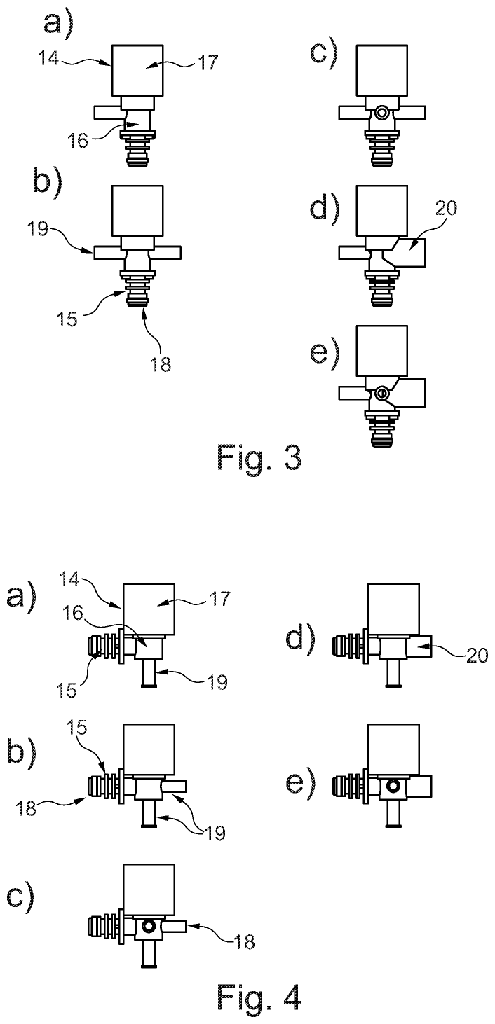

FIGS. 3 and 4 show a snap-in valve 14 in various configurations, which is provided for installation at the coupling receptacle (port) 6. The snap-in valve 14 is preferably a solenoid valve as an example of a hydraulic component. The snap-in valve 14 is suitable and provided to be inserted into the coupling receptacle 6 of the valve island 1 via a coupling portion 15 of the snap-in valve 14, in order to be connected to the fluid flow passage 3 of the base body 2, in a radially tight and form-fitting manner. The snap-in valve 14 has a valve seat 16 which can be opened and closed via a movable valve body in order to open and/or close the internal fluid flow passage of the snap-in valve 14. The snap-in valve 14 further comprises, as in the valve island 1 according to Fig.1 and 2, an electrical coil element 17.

The coupling portion 15 of the snap-in valve 14 in FIGS. 3 and 4 tapers in a frustoconical shape from a bottom portion to a flattened tip portion. In an axial (longitudinal) direction of the coupling portion 15, the coupling portion 15 has radial recesses extending radially inward to an outer circumference of the fluid flow passage of the coupling portion 15. At the circumference of the tip portion, an O-ring 18 is arranged as a sealing element, which is suitable and provided to form the connection of a fluid flow passage formed to the valve island 1 in a radially sealing manner.

The various configurations of the snap-in valve 14 include one or a plurality of spouts 19 connected to the internal fluid flow passage of the snap-in valve 14 and provided for connection of external hydraulic lines or tubes. Further, in addition to the one or more spouts 19, some of the configurations of the snap-in valve 14 may include a connecting portion 20 suitable and provided for connection/coupling of, for example, sensors or indicating instruments.

The configurations a) to e) in FIG. 3 of the snap-in valve 14 have a coupling portion 15 that is formed axially to the coil element 17. The configurations of the snap-in valve 14 in FIG. 4 a) to e) differ from those in FIG. 3 essentially in that a coupling portion 15 is formed here at an angle of 90° to the coil element 17.

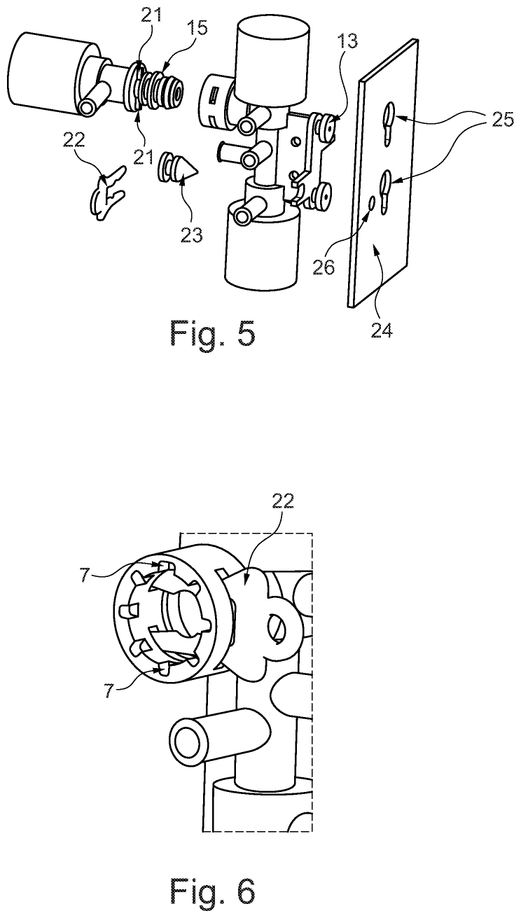

FIG. 5 is a schematic representation of the individual parts of a configuration of the valve island 1 according to the first configuration example (in an exploded view) with the snap-in valve 14, a mounting clip 22 as mounting/axial securing element, a valve locking element 23 and a connecting portion/holding plate 24 of the carrier/valve-island holder as mounting interface.

A bottom portion of the coupling portion 15 of the snap-in valve 14 (corresponding to an end disk axially bounding the coupling portion 15) has a circular front surface facing the coupling portion 15 in the axial direction, and in the center of which the fluid flow passage of the snap-in valve 14 is arranged with a diameter smaller than that of the circular front surface. Two projections 21 are arranged on the circular front surface, which project in the axial direction from the circular front surface toward the coupling portion 15 and also extend in the radial direction, as shown in FIG. 5, respectively above and below the fluid flow passage of the snap-in valve 14 in the upward and downward directions. In the connected state (snap-in valve 14 is inserted into port 6), the projections 21 on the coupling portion 15 engage in the respective depression 7 of the coupling receptacle/port 6. This prevents the snap-in valve 14 from rotating axially in relation to the valve island 1.

The connecting portion 24 of the carrier has two female connection receptacles 25, which are arranged at a distance one above the other as shown in FIG. 5, are designed in the form of keyholes and are suitable and provided for fastening the respective two male connection elements 13 of the valve island 1 to the connecting portion 24 of the carrier, for example by hooking them into the female connection receptacles 25.

The valve locking element 23 is conical and suitable for locking the valve island 1 to the connecting portion 24 of the carrier. In order to lock the valve island 1, the valve locking element 23 is inserted/pressed through a U-shaped section of the connecting base plate 12 of the valve island 1 (see FIG. 2), which protrudes laterally from the longitudinal extension of the connecting base plate 12 (see FIG. 5), and through a bore 26 provided for this purpose on the carrier, and is locked/anchored by radial expansion effect.

FIG. 6 shows an enlarged view of a coupling receptacle 6 of the valve island 1 with a mounting clip 22 according to the first configuration example. The mounting clip 22 has a base portion from the upper and lower ends of which one leg each extends at a 90° angle, which together with the base portion form a U-shape and are parallel. The legs of the mounting clip 22 are inserted into locking openings of the coupling receptacle 6 from the radial direction for mounting the snap-in valve 14, and partially embrace the fluid flow passage of the coupling portion 15 inserted therein in the radial recesses. The coupling portion 15 is secured in the axial direction by the spring force of the legs of the mounting clip 22.

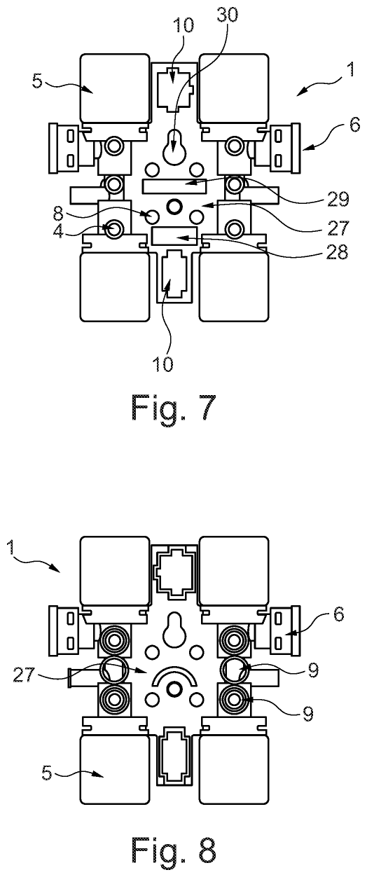

FIGS. 7 and 8 show a schematic representation of a front side and an mounting side of the valve island 1 according to a second configuration example of the present disclosure.

The valve island 1 of the second configuration example differs from the valve island 1 of the first configuration example by the base body 27, on which a further (second), independent fluid flow passage is formed, which is preferably connected in one piece of material to the base body 27 axially parallel to the first fluid flow passage on the same or an opposite side of the base body. In other words, the base body 27 serves, among other things, as a middle or connecting part between the parallel-spaced fluid flow passages.

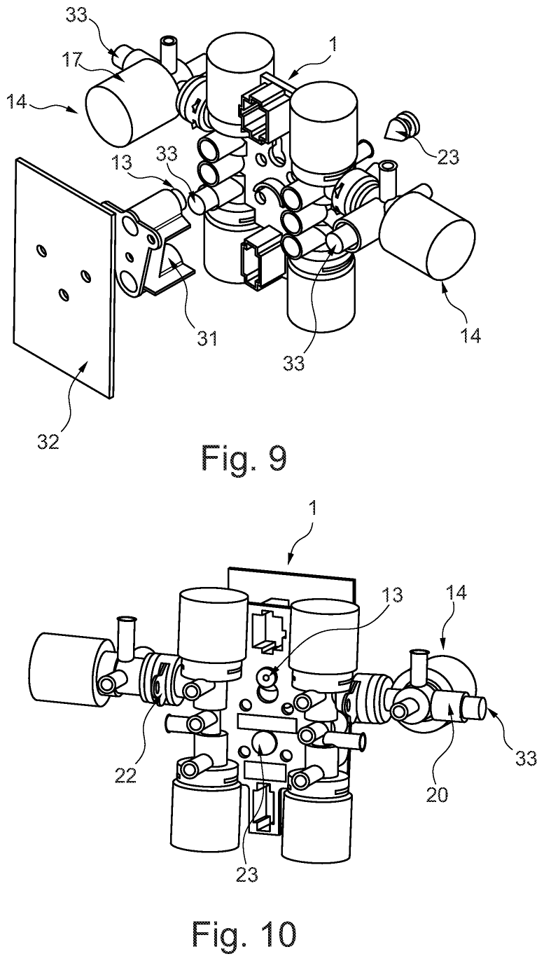

The two fluid flow passages according to FIGS. 7 and 8 are each formed substantially identically to the fluid flow passage 3 according to the first configuration example. That is, each of the two fluid flow passages of the second configuration example has two coil elements 5 with valve seat placed on the front side, a coupling receptacle/port 6 for, e.g. a further (plug-in) valve, a number (preferably four) of spouts 4 for separate lines and other hydraulic components, and a number (preferably three) of fluidic connecting portions 9, all of which are formed integrally with the two fluid flow passages. The respective coupling receptacles 6 are arranged laterally of the base body 27 in such a way that they face in opposite directions and are thus each accessible from laterally outside the base body 27 without interfering with each other. Three of the number of spouts 4 are arranged on one (front) side and one of the number of spouts 4 is arranged laterally (parallel to the respective coupling receptacle 6) of the base body 27. The number of fluidic connecting portions 9 are all arranged on the mounting side, i.e. on the longitudinal side of the respective fluid flow channels facing away from the spouts 4. The base body 27 has a number, preferably four bores 8 and a number, preferably two plug windows 10. The front side of the base body 27 is provided with a data matrix code 28 and a marking for the type label 29. The base body 27 has a female connection receptacle 30 similar to that described in the first configuration example, but which is formed in a rotated keyhole shape and is suitable and provided to be hooked into a male connection element 13 arranged on an additional mounting adapter 31 (see FIG. 9) for attachment to the mounting adapter 31, wherein the mounting adapter 31 is in turn provided for connection to a holder/holding plate/carrier 32.

FIG. 9 shows a schematic representation of the individual parts of a configuration of the valve island 1 of the second configuration example with two snap-in valves 14 connected (optionally) to the coupling receptacles 6, the valve locking element (expansion clip/pin) 23, the mounting adapter 31 and the carrier or, respectively, the connecting portion 32 of the carrier as mounting interface.

In this case, the mounting adapter 31 serves as a spacer to create a distance between the valve island 1 and the connecting portion 32 of the carrier. This is necessary due to the special design of the mounting side (the side facing the carrier) of the valve island 1, on which the fluidic connecting portions 9 are arranged in the second configuration example. Likewise, the distance is required for one of the two stretch valves connected to the coupling receptacle 6, in which the spool body 17 of the snap-in valve is angled 90° to the coupling portion 15 of the snap-in valve in the direction towards the carrier.

On a side facing the valve island, the mounting adapter 31 has at least one of the male connection elements (hooks) 13 described above, unlike the valve island 1 of the first configuration example, in which the connecting base plate 12 of the base body 2 has the male connection elements 13. The side of the mounting adapter 31 facing the carrier further has a number of (preferably three) bores suitable for mounting on the carrier, on which congruently correspondingly similar bores are provided.

FIG. 10 shows a schematic representation of a front view of an overall configuration of the valve island 1 of the second configuration example. In this illustration, the valve island 1 is mounted on the carrier via the mounting adapter 31 (not shown in this Fig.) and locked via the expanding mandrel 23 (see FIG. 5). Two snap-in valves 14 are connected/inserted to the coupling receptacles 6 of the base body 27 and fastened/axially fixed via a mounting clip/splint 22. A sensor 33 is optionally inserted in the fluidic connecting portions 20 of the snap-in valve 14 and one of the fluidic connecting portions of the base body 27.

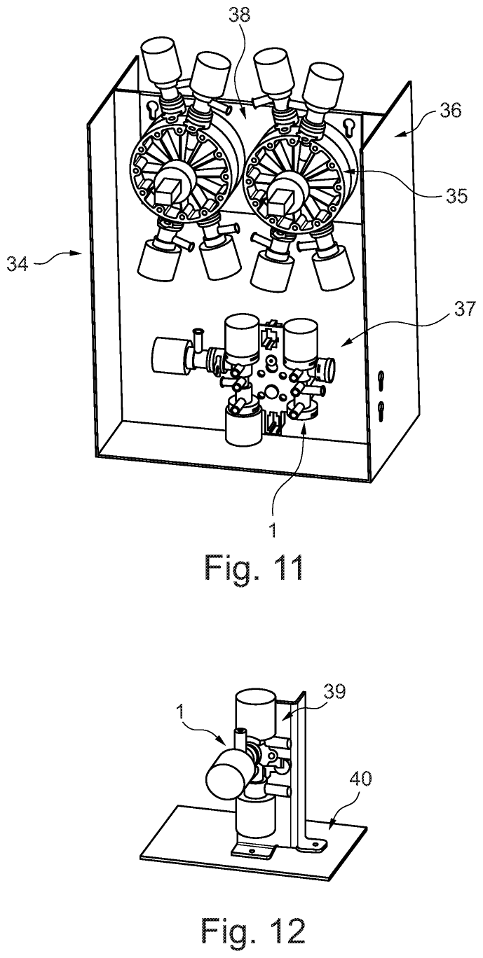

FIG. 11 is a schematic representation of an example of a hydraulic assembly 34 in a dialysis machine with balance chambers 35 and the valve island 1 according to the second configuration example.

An assembly tower (housing portion of the dialysis machine) 36 preferably has a mounting plate 37 that serves as a mounting surface or mounting plane/carrier for the valve island 1 and possibly also for the balance chambers 35. The mounting plate 37 is provided in a U-shaped frame of the assembly tower 36. The space defined/surrounded by the assembly tower 36 is divided by the mounting plate 37 into a front and a rear space section. The valve island 1 is mounted to the mounting plate 37 in an area, according to FIG. 11, below the balance chambers 35.

The balance chambers 35 are mounted on a plate-like sub-assembly 38, which is mounted in a planar manner in the mounting plate 37 to form a common plane with the mounting plate 37.

FIG. 12 shows a schematic representation of the valve island 1 according to the first configuration example, which is mounted on a mounting angle 39, which in turn is attached to an insertion sheet 40. Due to the mounting via the mounting angle 39, the valve island 1 is oriented at an angle of 90° with respect to the insertion sheet 40. The insertion sheet 40 is suitable and provided for insertion or sliding into the hydraulic assembly, for example the assembly tower preferably of a dialysis machine.

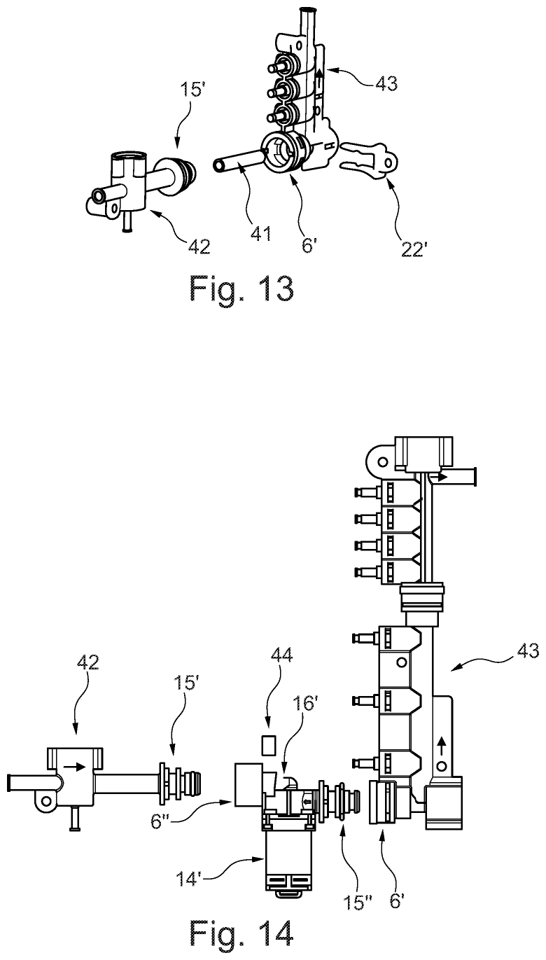

FIG. 13 shows a schematic representation of the individual parts of a configuration of a sub-assembly with a mixing element 41 integrated in a fluid flow passage of a check valve 42. The check valve 42 with the integrated mixing element 41 is suitable and provided to be inserted into a coupling receptacle 6′ of the sub-assembly or into the coupling receptacle 6 of the valve island 1 (see e.g. FIG. 1) via a coupling portion 15′ of the check valve 42 in order to be radially tightly and form-fittingly connected to a fluid flow passage of a conductivity measuring sensor 43 or the valve island 1. As already shown in FIG. 6, the connection is suitable for being axially fastened via a mounting clip 22′ as a mounting element.

FIG. 14 shows a schematic representation of the individual parts of a further configuration of a sub-assembly with a mixing element 44, which is integrated into the valve seat 16′ of a snap-in valve 14′. The snap-in valve 14′ with the integrated mixing element 44 is suitable and provided to be inserted into a coupling receptacle 6′ of the sub-assembly or into the coupling receptacle 6 of the valve island 1 (see e.g. FIG. 1) via a coupling portion 15″ of the snap-in valve 14′ in order to be connected with a fluid flow passage of a conductivity measuring sensor 43 or the valve island 1 in a radially tight and form-fitting manner. The check valve 42 (here without mixing element) is suitable and provided to be inserted into a coupling receptacle 6″ of the snap-in valve 14′ via the coupling portion 15′ of the check valve 42 in order to be connected to the snap-in valve 14′ in a radially tight and form-fitting manner. FIG. 14 thus shows the following connection arrangement: check valve 42, snap-in valve 14′ with integrated mixing element 41′ and conductivity measuring sensor 43.

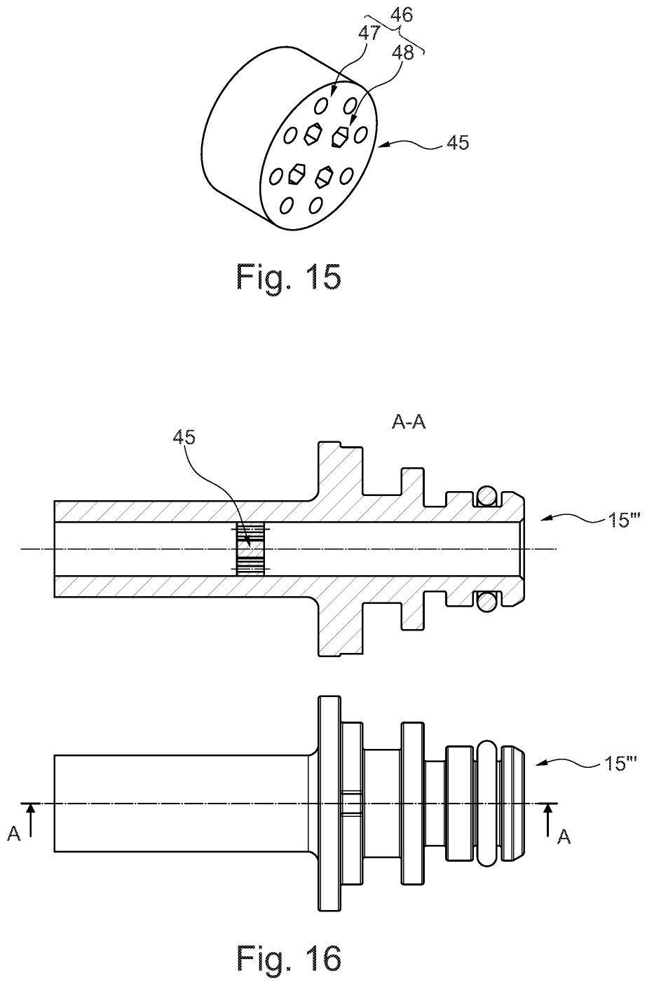

FIG. 15 shows a schematic representation of a further variation of a mixing element as a mixing element 45. A plurality of flow passages 46 are arranged parallel to each other and parallel to a central axis A of the mixing element 45 within the fully cylindrical mixing element 41″. Eight flow passages with a circular cross-section 47 are arranged in the immediate vicinity of the outer circumference of the mixing element 45 at uniform intervals in the circumferential direction. Four flow passages with a hexagonal cross-section 48 are arranged in the immediate vicinity of the center axis A in the circumferential direction at uniform intervals.

FIG. 16 shows a cross-sectional view along a plane A-A of the center axis A of the mixing element 45, which is integrally connected to a coupling portion 15′″ by injection molding or additive manufacturing. The mixing element 41″ can be overmolded with the coupling portion 15′″ as a pre-molded part.

1. A valve island for a hydraulic assembly for an extracorporeal blood treatment machine with a base body in/on which at least one fluid flow passage comprising a tube is formed/integrally arranged,
wherein the base body is manufactured by additive manufacturing or injection molding, and
wherein a valve portion is arranged at at least one axial end of the at least one fluid flow passage.

2. The valve island according to claim 1, wherein the valve portion is integrally formed with the base body.

3. The valve island according to claim 1, wherein the valve portion is rotatably arranged on the base body in steps.

4. The valve island according to claim 1, wherein at least one coupling receptacle is formed integrally with the base body, via which a hydraulic component is connectable fluidically and positively to the at least one fluid flow passage.

5. The valve island according to claim 1, wherein at least one spout is formed integrally with the base body, via which a fluidic line can be fluidically connected to the at least one fluid flow passage.

6. The valve island according to claim 1, wherein at least one fluidic and/or mechanical connecting portion is formed integrally with the base body, via which a component is connectable fluidically and/or mechanically.

7. The valve island according to claim 1, wherein at least one connecting portion is formed on the base body, via which the valve island is connectable to a connecting portion of the carrier or a mounting adapter matched thereto.

8. The valve island according to claim 4, wherein the hydraulic component is a valve that is fluidically and positively connected to the at least one fluid flow passage via the coupling receptacle.

9. The valve island according to claim 8, wherein the valve comprises a coupling portion inserted into the coupling receptacle and secured therein via a mounting element.

10. The valve island according to claim 8, wherein a mixing element for mixing fluids is integrated in a fluid flow passage of the valve or a second valve.

11. The valve island according to claim 8, wherein a mixing element is integrated in a valve seat of the valve.

12. The valve island according to claim 4, wherein the hydraulic component is a mixing element that is fluidically and positively connected to a fluid flow passage or the at least one fluid flow passage via the coupling receptacle or a coupling receptacle.

13. The valve island according to claim 12, wherein the mixing element comprises a coupling portion inserted into the coupling receptacle and secured therein via a mounting element.

14. A hydraulic assembly for an extracorporeal blood treatment machine with a carrier on which at least one valve island according to claim 1 is arranged indirectly or directly.

15. The hydraulic assembly according to claim 14, wherein the carrier includes a surface on which the at least one valve island is or are disposed.

16. The hydraulic assembly according to claim 14, wherein the at least one valve island is mountable or dismountable from a common side.

17. The hydraulic assembly according to claim 14, wherein the carrier has at least one adapted connecting portion connected to a connecting portion of the base body.

18. The hydraulic assembly according to claim 14, further comprising a mounting adapter, wherein the mounting adapter has at least one adapted connecting portion connected to the connecting portion of the base body and at least one secondary connecting portion connected to the carrier.

19. A dialysis machine comprising a hydraulic assembly according to claim 14.
